(19) United States
(12) Patent Application Publication
(10) Pub. No.: US 2006/0029597 A1
(43) Pub. Date: Feb. 9, 2006

(54) IgA antibody protein as a cytotoxic drug

(75) Inventors: Koteswara Chintalacharuvu, Torrance, CA (US); Manuel Penichet, Los Angeles, CA (US); Sherie Morrison, Los Angeles, CA (US)

Correspondence Address: PATTON BOGGS LLP, 8484 WESTPARK DRIVE, SUITE 900, MCLEAN, VA, 22102, US

(73) Assignee: Chimeric Technologies, Los Angeles, CA (US)

(21) Appl. No.: 11/094,302

(22) Filed: Mar. 31, 2005

Related U.S. Application Data
(60) Provisional application No. 60/557,696, filed on Mar. 31, 2004.

(51) Int. Cl. A61K 39/395; C12N 5/06; C07K 16/44

(52) U.S. Cl. 424/133.1; 530/387.3; 435/328

(57) ABSTRACT

Novel compounds are disclosed. The compounds are useful for inducing apoptosis, cell death and/or inhibiting proliferation of cells. Also disclosed are pharmaceutical compositions and methods associated with the compounds.

The present application claims priority to U.S. Provisional Application Ser. No. 60/557,696, filed Mar. 31, 2004, which is herein incorporated by reference in its entirety.

### FIELD OF THE INVENTION

The present invention relates generally to chimeric antibodies useful for causing apoptosis, cell death and/or inhibition of proliferation of a wide variety of cell populations.

### BACKGROUND OF THE INVENTION

Multiple myeloma (MM) is an incurable monoclonal proliferative disorder of malignant plasma cells affecting more than 14,000 persons in the United States per year, and accounting for 2% of cancer related deaths. Despite advances in medicine, the five-year survival of patients with myeloma has shown little improvement since the 1970's. Although conventional therapy of multiple myeloma with oral melphalan and prednisone can effect remissions in approximately 40% of patients, the disease remains incurable, with a median overall survival of only 30 to 36 months. Alexanian and Dimopoulos, N. Engl. J. Med. 330:484 (1994); Imamura et al., Int. J. Hematol. 59:113 (1994); Kyle, Cancer 72:3489 (1993); Vescio et al., Blood 93:1858 (1999). Multi-agent, conventional-dose chemotherapy regimens can result in improved response rates, however, there has been no significant improvement in the length of survival for patients treated with these multi-drug regimens compared with standard doses of melphalan and prednisone.

High-dose therapy (HDT) followed by autologous bone marrow transplantation can improve both response rates and overall survival. Fermand et al., Blood 82:2005 (1993); Harousseau et al., Blood 79:2827 (1992); Jagannath et al., Oncology (Huntingt) 8:89 (1994); Attal et al., N. Engl. J., Med. 335:91 (1996). Nevertheless the majority of multiple myeloma patients treated with autologous bone marrow transplantation show evidence of progressive disease within 3 years. Malignant cells have been detected in bone marrow harvests, peripheral blood and in the leukapheresis products used for transplantation of patients with multiple myeloma (Gertz et al., Bone Marrow Transplant 19:337 (1997); Shpall et al., Blood 90:4313 (1997); Lopez and Beaujean, Baillieres Best Pract. Res. Clin. Haematol. 12:71 (1999); Baynes et al., Clin. Chem. 46:1239 (2000)) and may be responsible for the failure to obtain long-term patient survival. More effective methods of removing tumor cells before autologous transplantation are required.

Because of their accessibility, hematopoietic malignancies are excellent targets for antibody-mediated therapy. Rituxan™, the first FDA approved antibody against cancers, has been demonstrated to be very effective in the treatment of CD20 positive B-cell non-Hodgkin's lymphoma. However, plasma cells do not express CD20 on their surface and multiple myelomas are not sensitive to Rituxan™. Thus, additional antibody-based strategies for treatment of multiple myeloma patients are urgently needed.

The primary function of serum transferrin (Tf) is to bind iron and after interacting with the transferrin receptor (TfR) present on the surface of cells and transport it into cells. Dowlati et al., Br. J. Cancer 75:1802 (1997). After binding its receptor on the cell surface, Tf is internalized into an acidic compartment where iron dissociates and the apo-Tf is returned to the cell surface where ligand-receptor dissociation occurs. It has also been proposed that the TfR serves a role as a growth factor independent of its function as a transporter of iron. May and Cuatrecasas, J. Membr. Biol. 88:205 (1985). In fact, Tf is considered to be an autocrine regulator of cell proliferation in malignant tumor cells. Dowlati et al. (1997); Shapiro and Wagner, In Vitro Cell Dev. Biol. 25:650 (1989); Vostrejs et al., J. Clin. Invest. 82:331 (1988). High-level expression of the TfR has been identified on many hematopoietic malignancies such as lymphomas (Habelshaw et al., Lancet 1:498 (1983)), leukemias (Beguin et al., Leukemia 7:2019 (1993)) and myelomas (Jefferies et al., Immunology 54:333 (1985); Lesley et al., Exp. Cell. Res. 150:400 (1984)). Because of its malignant phenotype, myeloma cells express much higher level of TfR than normal tissues such as hematopoietic stem cells. Jefferies et al. (1985); Lesley et al. (1985). In fact, the TfR is expressed at very low level in early stem cells (Gross et al., Eur. J. Haematol 59:318 (1997)) and there are subsets of bone marrow and peripheral blood stem cells that do not express the TfR at all. Gross et al. (1997); Bender et al., Clin. Immunol. Immunopathol. 70:10 (1994).

Cytotoxic compounds may be conjugated with transferrin or antibodies against transferrin to successfully target and eliminated certain cancer cells in vitro and in vivo. A major concern is that the conjugates may be cytotoxic to the normal cells expressing the TfR. However, previous preclinical and clinical studies using toxins chemically conjugated to Tf have shown that the cytotoxicity was mainly directed to the tumor cells and that side effects of the treatment were minor or absent when the conjugate was administered locally (intratumoral administration) (Laske et al, Neurosurgery 41:1039 (1997); Laske et al., Nat. Med. 3:1362 (1997)) or systematically (Mayers et al., In Proceedings of the 89th Annual Meeting of the American Association for Cancer Research, New Orleans, La., USA, Mar. 28-Apr. 1, 1998. 63 (1998); U.S. Pat. No. 5,393,737).

TfR may be utilized as a specific target of antibody based therapy. Several mouse or rat monoclonal antibodies specific for the mouse, rat, or human TfR have been developed Jefferies et al. (1985); Lesley et al., Exp. Cell. Res. 182:215 (1989); White et al., Cancer Res. 50:6295 (1990). One example is the murine IgA monoclonal antibody named 42/6 specific for human TfR. 42/6 was able to significantly inhibit the proliferation of several human malignant cell lines (Trowbridge et al., Methods Enzymol. 147:265 (1987); Trowbridge and Lopez, Proc. Natl. Acad. Sci. (USA) 79:1175 (1982); Taetle et al., Cancer. Res. 46:1759 (1986); Lesley and Schulte, Mol. Cell. Biol. 5:1814 (1985)) and led to a Phase I clinical trial (Brooks et al., Clin. Cancer Res. 1:1259 (1995)). The treatment was well tolerated, with several patients showing a mixed anti-tumor response. The three responding patients were all affected with hematopoietic cancers: follicular lymphoma, Hodgkin's disease, and chronic lymphocytic leukemia. Multiple myeloma patients were not included in this trial. This monoclonal antibody did not show a significant anti-tumor activity. The lack of significant therapeutic results was explained by the extremely rapid clearance of IgA in circulation compared to IgG antibodies and by the presence of human anti-mouse IgA antibodies (HAMA) that were detected in several patients. Brooks et al. (1995).

Since TfRs are overexpressed on malignant cells, an anti-TfR avidin fusion protein may be used to transport cytotoxic agents into tumor cells. One example is an anti-human TfR IgG3-Av (avidin) fusion protein constructed by substituting the variable regions of the heavy and light chains of anti-dansyl IgG3-Av with the variable regions of anti-human TfR IgG1 monoclonal antibody 128.1. Ng et al., Proc. Natl. Acad. Sci. USA 99:10706 (2002). This fusion protein was shown to inhibit the growth of a human erythtoleukemia cell line and eight human malignant plasma cell lines. Inhibition was observed when the anti-rat variable regions and the avidin moiety were present on the same molecule. The fusion protein was able to inhibit the growth of two cancer cell lines of hematopoietic linage. Ng et al. (2002). Studies have shown that anti-TfR IgG3 does not dimerize, suggesting that non-covalent interactions among the avidin molecules lead to dimerization and that this results in the anti-proliferative/apoptotic activity of anti-TfR IgG3-Av.

The high levels of expression of TfR in cancer cells, which may be up to 100 fold higher than the average of normal cells (Prost et al., Int. J. Oncol. 13:871 (1998); Shinohara et al., Int. J. Oncol. 17:643 (2000)), its extracellular accessibility, its ability to internalize, and its central role in the cellular pathology of human cancer, make this receptor an attractive target for the therapy of cancer.

### SUMMARY OF THE INVENTION

The present invention pertains to methods of causing apoptosis, cell death or inhibiting of proliferation of cells expressing TfR. This invention is based on the development of a novel chimeric antibody with human constant regions with the ability to bind to human transferrin receptor. The present invention also pertains to therapeutic compositions for causing apoptosis, cell death or inhibiting of proliferation of cells which express TfR.

According to one aspect, the present invention provides a compound comprising: 1) a heavy chain constant region and a light chain constant region of a human antibody; and 2) a heavy chain variable region and a light chain variable region that recognizes the human transferrin receptor. In one embodiment, the heavy chain constant region and the light chain constant region comprise human IgA. In another embodiment, the heavy chain constant region and the light chain constant region comprise human IgM. In yet another embodiment, the heavy chain constant region and the light chain constant region comprise human polymeric IgG. In another aspect, the heavy chain variable region and light chain variable region are murine.

In an additional aspect, the present invention provides a pharmaceutical composition comprising the compound of above in combination with a pharmaceutically acceptable carrier. In another aspect, the invention provides a method of treating a malignancy that expresses the human transferrin receptor in an individual, comprising administering the pharmaceutical composition described above to the individual, in a therapeutically effective amount. In one embodiment, the malignancy is selected from the group consisting of multiple myeloma, leukemia and lymphoma.

The present invention also provides a method of causing apoptosis or cell death in cells expressing the human transferrin receptor, comprising contacting the cell with the compound described previously. In one aspect, the heavy chain constant region and the light chain constant region of the compound comprise human IgA. In an additional aspect, the heavy chain constant region and the light chain constant region of the compound comprise human IgM. In yet another aspect, the heavy chain constant region and the light chain constant region of the compound comprise human polymeric IgG. In an additional embodiment, the heavy chain variable region and light chain variable region of the compound are murine. In yet another embodiment, the cells comprise malignant cells. In one aspect, the malignant cells are selected from the group consisting of multiple myeloma cells, leukemia cells and lymphoma cells.

The invention further provides a therapeutic composition for causing apoptosis or cell death in cells expressing the transferrin receptor on their surface, said composition comprising the compound as described previously. In one embodiment, the heavy chain constant region and the light chain constant region of the compound comprise human IgA. In another embodiment, the heavy chain constant region and the light chain constant region of the compound comprise human IgM. In an additional embodiment, the heavy chain constant region and the light chain constant region of the compound comprise human polymeric IgG In still another embodiment, the heavy chain variable region and light chain variable region of the compound are murine. In one aspect, the cells comprise malignant cells. In another aspect, the malignant cells are selected from the group consisting of multiple myeloma cells, leukemia cells and lymphoma cells.

The present invention further provides a method of inhibiting proliferation of cells expressing the human transferrin receptor, comprising contacting said cell with the compound as described above. In one embodiment, the heavy chain constant region and the light chain constant region of the compound comprise human IgA. In another embodiment, the heavy chain constant region and the light chain constant region of the compound comprise human IgM. In yet another embodiment, the heavy chain constant region and the light chain constant region of the compound comprise human polymeric IgG. In one aspect, the heavy chain variable region and light chain variable region of the compound are murine. In another aspect, the cells comprise malignant cells. In a further aspect, the malignant cells are selected from the group consisting of multiple myeloma cells, leukemia cells and lymphoma cells.

In addition, the present invention provides a therapeutic composition for inhibiting proliferation of cells expressing the transferrin receptor on their surface, said composition comprising the compound described previously. In one embodiment, the heavy chain constant region and the light chain constant region of the compound comprise human IgA. In another embodiment, the heavy chain constant region and the light chain constant region of the compound comprise human IgM. In a further embodiment, the heavy chain constant region and the light chain constant region of the compound comprise human polymeric IgG. In yet another embodiment, the heavy chain variable region and light chain variable region of the compound are murine. In another aspect, the cells comprise malignant cells. In a further aspect, the malignancy cells are selected from the group consisting of multiple myeloma cells, leukemia cells and lymphoma cells.

The invention further provides a method for autologous hematopoietic cell transplantation in a subject suffering from multiple myeloma, the method comprising: (1) removing the hematopoietic progenitor cell population from the subject; (2) treating the cell population with the compound described above; and (3) transplanting the treated cell population from step (2) into the subject. In one embodiment, the heavy chain constant region and the light chain constant region of the compound comprise human IgA. In another embodiment, the heavy chain constant region and the light chain constant region of the compound comprise human IgM. In a further embodiment, the heavy chain constant region and the light chain constant region of the compound comprise human polymeric IgG. In a further aspect, the heavy chain variable legion and light chain variable region of the compound are murine.

### BRIEF DESCRIPTION OF THE DRAWINGS

FIGS. 1A-1C are a schematic representation of IgG, monomeric IgA and dimeric IgA, respectively.

FIGS. 2A and 2B are SDS-PAGE analysis of non-reduced and reduced chimeric anti-TfR IgA, respectively.

FIG. 3 is a plot of cells analyzed by flow cytometry indicating that chimeric anti-TfR IgA binds specifically to TfR on K562 cells.

FIGS. 4A and 4B are plots of antibody concentration versus proliferation valises indicating that anti-TfR IgA inhibits proliferation of human ARH-77 cells.

FIG. 5A-5B are a plot of cells analyzed by flow cytometry indicating that anti-TfR IgA induces apoptosis in human ARH-77 cells at different concentrations.

FIG. 6 is a plot of antibody concentration versus proliferation values indicating that anti-TfR IgA antibody exhibits antiproliferative activity on human ARH-77 cells.

FIG. 7 is a plot of antibody concentration versus proliferation values indicating that anti-TfR IgA inhibited proliferation of ARH-77 and IM-9 hematopoietic cancer cells.

FIGS. 8A and 8B are graphical depictions of anti-TfR IgA versus percent of control, indicating that anti-human transferrin receptor IgA induces apoptosis in the hematopoietic cancer cell line ARH-77.

FIGS. 9A and 9B are graphical depictions of anti-TfR IgA versus percent of control, indicating that anti-human transferrin receptor IgA induces apoptosis in the hematopoietic cancer cell line IM-9.

### DETAILED DESCRIPTION OF THE INVENTION

This invention is based, in part, on the development of a novel chimeric antibody with human constant regions with the ability to bind to human transferrin receptor. In one embodiment, the chimeric antibody is a polymeric IgA with a minimum of four binding sites, which can be used as a cytotoxic agent to treat cell populations both in vivo and in vitro to cause apoptosis, cell death and/or inhibit cell proliferation. In an additional embodiment, the chimeric antibody o the present invention is not bound to a cytotoxic compound.

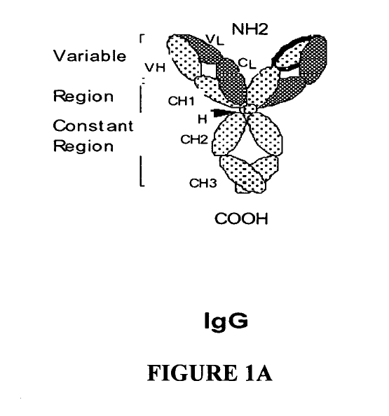

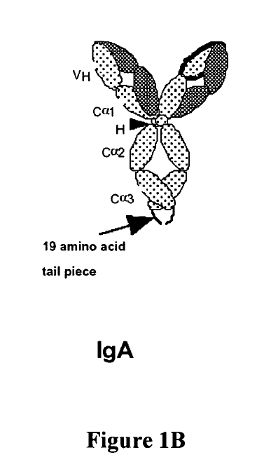

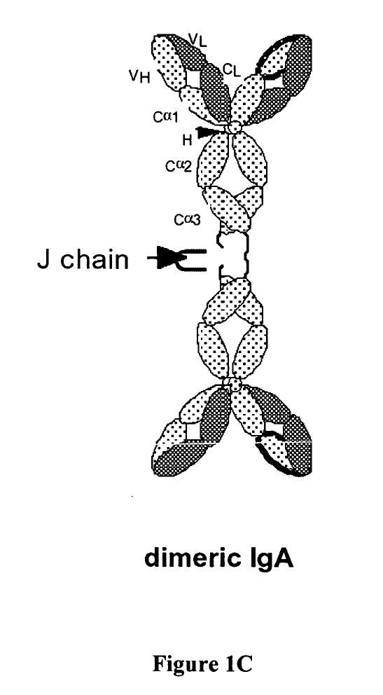

Antibodies are composed of two light and two heavy chain molecules. These chains are divided into domains of structural and functional homology. The variable domains of both the light (V_{L}) and the heavy (V_{H}) chains determine recognition and specificity. The constant region domains of light (C_{L}) and heavy (C_{H}) chains confer important biological properties such as antibody chain association, secretion, transplacental mobility and complement binding. A schematic representation of IgG, monomeric IgA and dimeric IgA is shown in FIGS. 1A and 1B. IgG and monomeric IgA contain only two antigen binding sites. In contrast, dimeric IgA contains four binding sites.

In one embodiment of the present invention, the human constant regions of the anti-human TfR chimeric antibody comprise IgA. One characteristic of IgA is its presence as polymers with dimers as the predominant form (FIG. 1B). In contrast to IgG, IgA H chain has a 19 amino acid extension at the carboxy terminus of the C_{H}3 exon with a penultimate cysteine required for polymer formation. Like IgG, monomeric IgA consists of a unit H₂L₂. The assembly of dimeric IgA is initiated with the formation of H₂L₂monomer units. Chintalacharuvu and Morrison, J. Immonol. 157:3443 (1996); Chintalacharuvu et al., J. Immunol. 169(9):5072-7 (2002); Chuang and Morrison, J. Immunol. 158:724 (1997). The penultimate cysteine in the tail-piece of one α chain forms a disulfide bond with J chain, which in turn forms a disulfide bond with the H chain of a second monomeric subunit. Atkin et al., J. Immunol. 157:156 (1996); Bastian et al., Biol. Chem. Hoppe Seyler 373:1255 (1992); Niles et al., Proc. Natl. Acad Sci. USA 92:2884 (1995). While IgG with the tailpiece of IgA can form polymers without J chain (Smith et al., J. of Immunol. 154:2226 (1995)), J chain is incorporated into the polymers of Igs containing C_{H}3 of IgA. Yoo et al., J. Biol. Chem. 274:33771 (1999). Some dimers are present in serum from mice deficient in J chain expression. Hendrickson et al, J. Immunol. 157:750 (1996).

Methodologies for producing chimeric antibodies are well know to those of skill in the art. For example, the light and heavy chains, or variable and constant regions, can be expressed separately, using, for example, separate plasmids. These can then be expressed, purified and assembled in vitro into complete antibodies; methodologies for accomplishing such assembly have been described.

The invention also provides that a nucleic acid molecule encodes the chimeric molecule. Expression vectors to produce mouse-human chimeric IgA1, IgA2m(1), IgA2m(2) and IgA2m(n) in mouse myeloma cells and Chinese hamster ovary (CHO) cells have been developed. Rifai et al., J. Exp. Med. 191:2171 (2000). These vectors were used in the present invention to transfect murine non-producing myeloma cells (results not shown). IgA was isolated from the resulting transfectants and analyzed by SDS-PAGE under non-reducing conditions. IgA1, IgA2m(2) and IgA(n) showed two predominant bands corresponding to dIgA and mIgA and minor bands corresponding to HL and dIgA lacking L chains (results not shown).

In the present invention, an anti-TfR α chain expression vector has been created and expressed it in mouse myeloma cells expressing the corresponding L chain (See Example 1). In one embodiment of the invention, the novel chimeric antibody contains the variable region of anti-TfR IgG3-Av, as described above.

The expression vectors are transfected into host cells for expression. Transfection vectors can be used in conjunction with the fusion protein cloning cassettes for expression of both the variable and constant regions. Electroporation is the one method for introducing DNA into host cells. Stable transfectomas are isolated using the selectable drug markers and culture supernatant is screened by ELISA. Cytoplasmic and secreted chimeric proteins are analyzed by SDS-PAGE under reducing and non-reducing conditions to verify expected molecular weight.

Recombinant genes, such as those producing the chimeric antibodies of the present invention, may also be introduced into viruses, such as adenovirus or herpes virus. Such viruses may be either defective or competent for replication. Recombinant viruses can be generated by transfection of plasmids into cells infected with virus.

A variety of host-vector systems may be utilized to express the protein-encoding sequence(s). The vector system must be compatible with the host cell used. Host-vector systems include but are not limited to bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria.

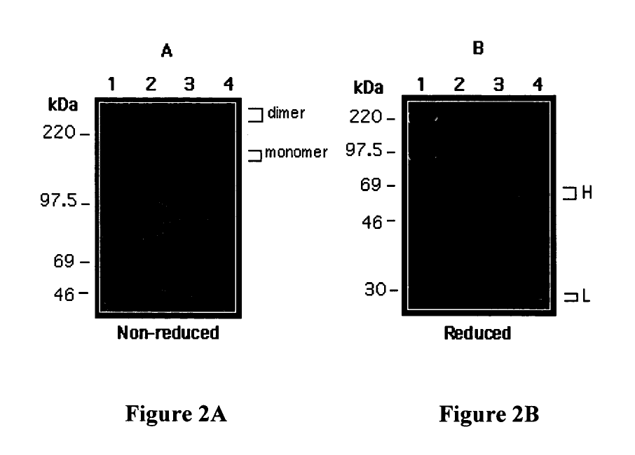

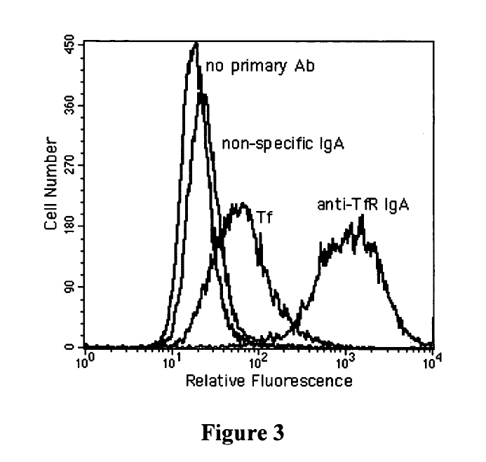

The IgA protein of the present invention was immunoprecipitated from culture supernatants and the anti-TfR IgA analyzed by SDS-PAGE under non-reducing conditions to determine the assembly and secretion of anti-human TfR dimeric IgA by the transfectants (See Example 2; FIG. 2B). IgA1, IgA2m(2) and IgA(n) showed two predominant bands corresponding to dIgA and mIgA and minor bands corresponding to HL and dIgA lacking L chains. The binding specificity of the antibody to TfR expressed on K562 human erythroleukemia cells is confirmed by flow cytometry (See Example 3; FIG. 3).

A 19 amino-acid sequence from the carboxy-terminus of IgA is responsible for the polymerization of IgA. The addition to the 19 amino-acids from IgA, a 19 amino-acid sequence from IgM will also lead to polymerization of antibody, antibody fragments or any ligands. These 19 amino acids of human IgA and IgG were grafted onto human IgG. It was found that this manipulation caused the IgG molecule to form polymers. This new IgG molecule, hereinafter referee to as “polymeric IgM” acts similarly to IgA and IgM, suggesting that the molecule would also exhibit anti-proliferative/apoptotic activity. Penichet and Morrison, Drug Devel. Res. 61:121-136 (2004). Therefore, in a further embodiment of the present invention, the human constant regions of the anti-TfR chimeric antibody comprise IgM or polymeric IgG. The methods and procedures for producing such hetero-molecules using recombinant antibody techniques have been published and are well known to those skilled in the art.

The anti-TfR dimeric chimeric protein of the present invention may then be purified. To produce mg quantities of antibody, cells are routinely expanded into roller-bottles and grown until the medium is exhausted. Alternatively, a small-scale hollow-fiber growth system can be used when larger quantities of proteins are required. Additionally, the transfectomas remain tumorogenic so that protein can be produced in BALB/c or SCID mice. (See, e.g., Example 8)

The antibodies of the invention having human constant region can be utilized for use, especially in humans, without negative immune reactions such as serum sickness or anaphylactic shock. The antibodies can also be utilized in immunodiagnostic assays and kits in detectably labeled form (e.g., enzymes, ¹²⁵I, ¹⁴C, fluorescent labels, etc.), or in immunmobilized form (on polymeric tubes, beads, etc.), in labeled form for in vivo imaging, wherein the label can be a radioactive emitter, or an NMR contrasting agent such as a carbon-13 nucleus, or an X-ray contrasting agent, such as a heavy metal nucleus. The antibodies can also be used for in vitro localization of the antigen by appropriate labeling.

The IgA protein is only an example of polymeric forms of immunoglobulins. The novel chimeric antibodies of the present invention are not limited to those with an IgA constant region. For example, IgM, polymeric IgG or any antibody in the form of polymers with specificity to any structures on cell surface will have such an effect. Exemplary cell surface structures may include proteins or carbohydrates, including growth factor receptors, transferrin receptors, and insulin receptors. Exemplary growth factor receptors include epidermal growth factor receptors, vascular endothelial growth factor receptor, an insulin-like growth factor receptor, platelet-derived growth factor receptor, transforming growth factor P receptor, fibroblast growth factor receptor, interleukin-2 receptor, interleukin-3 receptor, erythropoietin receptor, nerve growth factor receptor, brain-derived neurotrophic factor receptor, neurotrophinn-3 receptor, and neurotrophin-4 receptor.

In addition to polymeric antibodies and polymeric antibody fragments, receptor ligands or single chain Fvs (scFv) may be used as the targeting moiety provided that they exhibit specificity for a cell surface protein or carbohydrate. Exemplary non-antibody molecules include receptor ligands such as transferrin, insulin, epidermal growth factors, vascular endothelial growth factor, insulin-like growth factor, platelet-derived growth factor, transforming growth factor β, fibroblast growth factor, interleukin-2, interleukin-3 receptor, erythropoietin, nerve growth factor, brain-derived neurotrophic factor, neurotrophinn-3, and neurotrophin-4, and any scFv molecules specific for cell surface protein and/or growth factor receptors such as transferrin receptors, and insulin receptors. Exemplary growth factor receptors include epidermal growth factor receptors, vascular endothelial growth factor receptor, an insulin-like growth factor receptor, platelet-derived growth factor receptor, transforming growth factor β receptor, fibroblast growth factor receptor, interleukin-2 receptor, interleukin-3 receptor, erythropoietin receptor, nerve growth factor receptor, brain-derived neurotrophic factor receptor, neurotrophinn-3 receptor, and neurotrophin-4 receptor.

The present invention also pertains to methods of causing apoptosis, cell death or inhibiting of proliferation of cells expressing TfR. In one embodiment, the cells expressing TfR are malignant cells. In an additional embodiment, the malignant cells comprise multiple myeloma cells, leukemia cells, or lymphoma cells. Apoptosis is an active and programmed physiological process for eliminating superfluous, altered or malignant cells. The process is characterized by shrinkage of cells, segmentation of the nucleus, condensation and cleavage of DNA into domain-sized fragments, in most cells followed by internucleosomal degradation. The apoptotic cells fragment into membrane-enclosed apoptotic bodies. Neighboring cells and/or macrophages then phagocytose the dying cell. Cells can be analyzed for being apoptotic with agents staining DNA, which stains differently in normal and apoptotic cells.

Specifically, the invention provides a chimeric antibody for use as a cytotoxic drug that has significant anti-proliferative and pro-apoptotic potential on cells expressing TfR. As described above, the murine IgA monoclonal antibody 42/6 did not show significant anti-tumor activity as explained by rapid clearance of IgA in the circulation and by the presence of anti-mouse IgA antibodies in the patient. Because anti-TfR IgG3-Av with a dimeric structure (as with the murine IgA monoclonal antibody 42/6) may be at least partially responsible for the cytotoxic activity, the anti-human TfR chimeric antibody, as embodied in one aspect of the present invention, with the variable regions of anti-TfR IgG3-Av exhibits an anti-tumor activity although its mechanism of action may differ of that described for 42/6. In addition, since this embodiment of the novel molecule contains human constant regions, the chimeric antibody may have a considerably longer in vivo half-life (at least 4.7 days) (Delacroix et al., J. Clin. Invest. 71:358 (1983)) than that reported for murine IgA (6-22 hours) (Brooks et al. (1995); Rifai and Wu, Immunology 69:610 (1990); Vieira and Rajewsky, Eur. J. Immunol. 18:313 (1988); Meijer et al., J. Pharmacol. Exp. Ther. 300:346 (2002)). Moreover, the human Fc regions may enhance the ability of the anti-TfR to bind to Fc receptors present on T and B cells, monocytes and macrophages, neutrophils and eosinophils and NK cells and dendritic cells, thus enhancing tumor killing in vivo. Mota et al., Eur. J. Immunol. 33:2197 (2003). As a result, the chimeric antibodies of the present invention may inhibit the rapid proliferation of B anf T cells that overespress TfR. Finally, due to the presence of human constant regions, the anti-TfR chimeric antibody should overcome the human anti-mouse antibody response (HAMA), a response that is mainly elicited by the constant region of the antibody. Penichet and Morrison, Antibody Engineering. In Encyclopedia of Molecular Medicine (EMM). Thomas E. Creighton, ed. John Wiley & Son, Inc., New York. 2002, Vol. 1, pp. 214-216. In summary, anti-TfR IgA, as embodied in the present invention, should overcome the immunogenicity of murine IgA, have the right effector functions, and possess a much longer half-life in plasma (days vs. hours).

IM-9 and ARH-77 are human hematopoietic cell lines obtained from ATCC. Although IM-9 and AR-77 were isolated from a MM and plasma cell leukemia patient respectively, these cell lines have been shown to be Epstein Barr Virus (EBV) transformed B lymphoblastoid cell lines. Drexler et al., Leukemia 13:1601-07 (1999). However, when ARH-77 is injected into SCID mice, this cell line behaves like an authentic human MM with mice developing hypercalcemia, lytic bone lesions and hind limb paralysis. Gado et al., Haemotologica 86:227-236 (2001); Cruz et al., Exp. Hematol. 86:227-36 (2001). Examples 4 and 6 illustrate how the anti-TfR chimeric antibodies of the invention exhibit anti-proliferative activity on these two hematopoietic cell lines. Additionally, Examples 5 and 7 illustrate how the anti-TfR chimeric antibody presently described induces apoptosis and cell death in the same two hematopoietic cell lines.

The present invention also pertains to therapeutic compositions for causing apoptosis, cell death or inhibiting of proliferation of cells which express TfR. In one embodiment, the cells are malignant. The invention is directed to a potential site of malignant cell vulnerability: the overexpressed transferrin receptor. The novel antibody therapy targeted at a neoplastic plasma cell with sufficient anti-proliferative/pro-apoptotic potential alone, or in combination with other agents will make a significant clinical impact. The utility of this therapeutic invention is not be restricted to the elimination of malignant cells in vivo but can also be used for in vitro approaches. One significant use of the novel chimeric antibodies herein disclosed is the efficient purging of myeloma cells during ex vivo expansion of hematopoietic progenitor-cells for use in autologous transplantation in MM patients. The present invention is not restricted to MM, but rather may be applied to other hematopoietic malignancies such as leukemias and lymphomas. The recombinant antibodies of the present invention would not necessarily be a replacement for the conventional or non-conventional MM therapies described above, but instead may also provide an alternative therapy to be used in combination with other anti-cancer approaches.

The therapeutic compositions of the present invention may be used to treat cells in vitro. In one embodiment, the therapeutic composition is contacted with the cells of interest. The cells are exposed to the therapeutic compositions for a sufficient time to allow apoptosis, cell death and/or inhibition to occur. Exposure times will vary depending upon the concentration of the therapeutic agent, the particular cell type and the exposure conditions. Exposure times may vary from a few hours to a few days or more.

The present invention also pertains to methods of treatment (prophylactic and/or therapeutic) of a malignancy as described above, using the chimeric antibodies of the invention. As used herein, “treatment” refers to clinical intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, preventing metastasis, lowering the rate of disease progression, amelioration or palliation of the disease state, and remission or improved prognosis.

In one aspect of the invention, the novel chimeric antibodies are used to target the tumor cell or malignant cells expressing a human transferrin receptor. The chimeric antibodies in accordance with the present invention may be used in vivo to treat both liquid and solid tumors. The chimeric antibodies of the invention can be formulated in a pharmaceutical composition or agent with a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Common suitable pharmaceutical carriers are described in “Remington's Pharmaceutical Sciences” by E. W. Martin.

Accordingly, the pharmaceutical composition of the invention can be introduced parenterally, transmucosally, e.g., orally (per os), nasally or transdermally. Parental routes include intravenous, intra-arteriole, intramuscular, intradermal, subcutaneous, intraperitoneal, intraventricular and intracranial administration. Preferably, administration is directly into the cerebrospinal fluid, e.g., by a spinal tap.

In another embodiment, the therapeutic composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989). To reduce its systemic side effects, this may be a preferred method for introducing the composition.

In yet another embodiment, the therapeutic composition can be delivered in a controlled release system. For example, a polypeptide may be administered using intravenous infusion with a continuous pump, in a polymer matrix such as poly-lactic/glutamic acid (PLGA), a pellet containing a mixture of chlolesterol and the anti-amyloid peptide antibody composition (U.S. Pat. No. 5,554,601) implanted subcutaneously, an implantable osmotic pump, a transdermal patch, liposomes, or other modes of administration.

The pharmaceutical compositions of the invention may further comprise a therapeutically effective amount of the chimeric antibodies of the invention, preferably in respective proportions such as to provide a synergistic effect in the said prevention or treatment. A therapeutically effective amount of an pharmaceutical composition of the invention relates generally to the amount needed to achieve a therapeutic objective.

The novel chimeric antibodies of the present invention are also applicable to the purging of malignant plasma cells from biological samples, be it fluid or tissue samples. The purging of myeloma cells from a fluid sample is part of the invention and may be practiced by contacting a biological fluid suspected of comprising malignant plasma cells with a chimeric antibody of the invention (i.e., an antibody with a heavy chain constant region and a light chain constant region of a human antibody, and a heavy chain variable region and a light chain variable region that recognizes the human transferrin receptor) that is capable of selectively binding to and causing apoptosis or cell death of the malignant cells. This method may be utilized for purging unwanted cells ex vivo by extracting a biological sample from a patient, eliminating the malignant cells by apoptosis induced by the chimeric antibodies described herein and then replenishing the purged sample to the patient.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLE 1

### Production of Transfectants Producing Anti-TfR IgA

An EcoR V—Nhe I fragment from an IgG3 expression vector (Ng et al., (2002)) containing the H chain variable region coding for the anti-TfR was cloned into a mammalian expression vector containing the α H chain constant region. Using established methods, the expression vector was transfected into Sp2/0 myeloma cells producing anti-TfR L chain (Ng et al., (2002)). Transfectants were selected in 5 mM histidinol. After two weeks, the surviving colonies were screened for IgA production by ELISA.

To detect transfectants secreting IgA, microtiter plates coated with anti-human α H chain (Sigma Immuno. Chem., St. Louis, Mo.) were incubated overnight at 4° C. with 50 μL of supernatants from the 96-well plates containing transfectants. Bound IgA were detected by alkaline phosphatase conjugated goat antiserum to human K L chain (Sigma Immuno Chem., St. Louis, Mo.). Color was developed by adding 5 mg/ml of disodium p-nitrophenyl phosphate (Sigma Imm. Chem., St. Louis, Mo.) in diethanolamine buffer, pH 9.8. The wells exhibiting the strongest absorbance at 410 nm were subcloned and the subclones screened in a similar manner.

To detect transfectants secreting IgA, microtiter plates coated with anti-human a H chain (Sigma Immuno. Chem., St. Louis, Mo.) were incubated overnight at 4° C. with 50 l of supernatants from the 96-well plates containing transfectants. Bound IgA was detected by alkalin phosphatase conjugated goat antiserum to human κ L chain (Sigma Immuno. Chem., St. Louis, Mo.). Color was developed by adding 5 mg/ml of disodium p-nitrophenyl phosphate (Sigma Immuno. Chem., St. Louis, Mo.) in diethanolamine buffer, pH 9.8. The wells exhibiting the strongest absorbance.

Using established methods in the laboratory, a combination of biosynthetic labeling using ³⁵S-methionine, immunoprecipitation and SDS-PAGE analysis under reducing and non-reducing conditions were used to determine if a) the correct sized H and L chains are synthesized and b) if the anti-TfR antibodies are assembled and secreted as monomers and dimers (Rifai at al. (2000); Chintalacharuvu et al, J. Immunol. 152:5299 (1994); Chintalacharuvu et al. (1996); Chintalacharuvu et al. (2002)) (data not shown). Clones producing the highest quantities of IgA were expanded in IMDM containing 10% (v/v) BCS. To obtain homogeneous population of cells, cell lines were subcloned by limiting dilution technique. Positive clones were frozen to provide a continuous source of a well-characterized cell line.

### EXAMPLE 2

### Purification and Characterization of Anti-TfR IgA in Small Quantities

About 25 ml of culture supernatants from the transfectant was incubated for 1 hr at 4° C. with rabbit anti-α bound to Sepharose beads. Antibodies bound to Sepharose beads were pelleted by centrifuging at 13,000×g for 2 min and washed four times with 1 mL of phosphate buffer, pH 7.8 containing 0.45 M NaCl. Antibodies were eluted by incubating with 0.1 M glycine, pH 2.5 for 15 min on ice. The eluted antibodies were separated from the beads by centrifugation and the glycine buffer was neutralized using 2M Tris buffer, pH 8.0. The antibodies were dialyzed against PBS, pH 7.2 to remove the salt. Then the eluted antibodies were analyzed by SDS-PAGE in phosphate-buffered 5% polyacrylamide gels under reducing and non-reducing conditions (FIGS. 2A and 2B). The molecular weight standards are shown in Lane 1. Chimeric anti-TfR IgA is shown in Lane 4. Included for comparison are chimeric anti-dansyl IgG2 (150 kDa) (Lane 2) and chimeric anti-dansyl IgA1 (Lane 3).

A predominant band of 350 kDa corresponding to dimeric IgA and a minor band of 160 kDa corresponding to monomeric IgA were observed (FIG. 2A, Lane 4). When the eluted antibodies were reduced by incubating at 37° for 1 hr in the presence of 0.15 M 2-mercaptoethanol and analyzed by SDS-PAGE in Tris-Glycine buffered 12.5% polyacrylamide gels (FIG. 2B), a band of 60 kDa corresponding to the a H-chain and a 25 kDa band corresponding to κ L-chain were observed. Note that in these gels the J-chain is known to co-migrate with the L-chain.

### EXAMPLE 3

### Anti-Tfr IgA Binds Specifically to the Human Erythroleukemia Cell Line K562

To determine if the anti-TfR IgA bound to TfR specifically, K562 cells known to express TfR were incubated with either anti-TfR IgA or a nonspecific anti-dansyl IgA. The bound IgA was detected by incubating with anti-K conjugated to fluoresceine and analysis by flow cytometry (FIG. 3). K562 cells were incubated with 5 μg of anti-TfR IgA or non-specific IgA for 1 hr on ice. The cells were washed with PBS and incubated for 1 hr on ice with anti-κ conjugated to FITC and analyzed by flow cytometry. Almost 100% of the cells bound anti-TfR IgA, whereas there was no binding of anti-dansyl IgA. The ligand transferring conjugated to fluoresceine was used as a positive control. These results indicate that the anti-TfR IgA is functionally active in binding to K562 cells expressing TfR.

### EXAMPLE 4

### Anti-TfR IgA Inhibits Proliferation

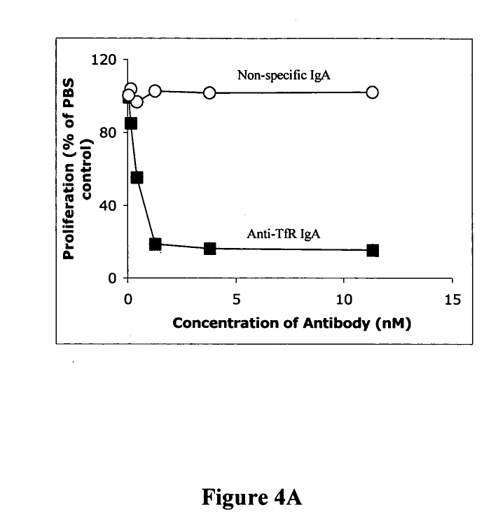

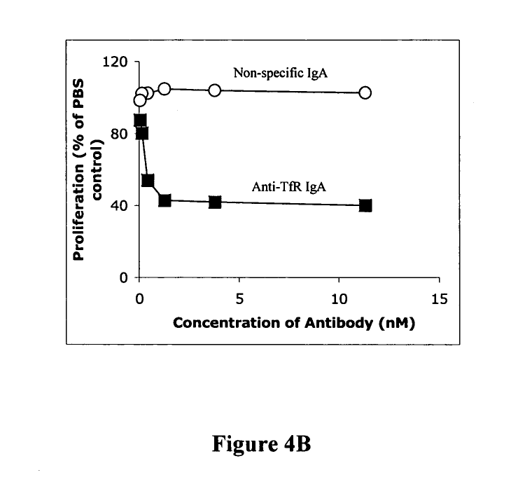

Human ARH-77 cells were treated with anti-TfR IgA at varying concentrations for 24 hours. The cells were then cultured in the presence of [³H]thymidine for an additional 24 hours. The cells were harvested and [³H]thymidine incorporation determined. The results are shown in FIGS. 4a and 4b. Each value (●) is a mean of four replicate values expressed as the % of control (cells treated with buffer alone, (∘)) mean. Results from two independent experiments are shown as FIG. 4A and FIG. 4B. The results indicate that anti-TfR IgA inhibits proliferation of human ARH-77 cells.

### EXAMPLE 5

### Anti-TfR IgA Induces Apoptosis

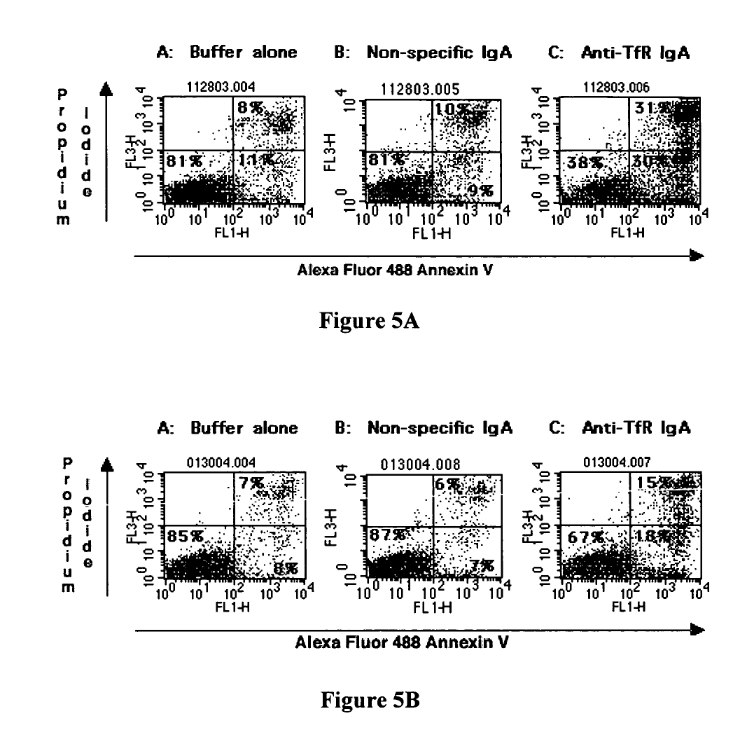

To determine if Anti-TfR IgA induces apoptosis, human ARH-77 cells were incubated with buffer alone (Panel A), 16.6 μg non-specific IgA (Panel B), or 16.6 μg anti-TfR IgA (Panel C) for 96 hours (FIG. 5A). The cells were then washed, stained with Alexa Fluor 488 Annexin V and PI, and analyzed by flow cytometry. The percentage of cells located in each quadrant is shown. Additionally, human ARH-77 cells were incubated with buffer alone (Panel A), 4.83 μg non-specific IgA (Panel B), or 4.83 μg anti-TfR IgA (Panel C) for 96 hours (FIG. 5B). The cells were then washed, stained with Alexa Fluor 488 Annexin V and PI, and analyzed by flow cytometry. The percentage of cells located in each quadrant is shown. Anti-TfR IgA induces apoptosis in human ARH-77 at varying concentrations.

### EXAMPLE 6

### Anti-human Transferrin Receptor IgA Exhibits Anti-Proliferative Activity on Hematopoietic Cancer Cell Lines ARH-77 and IM-9

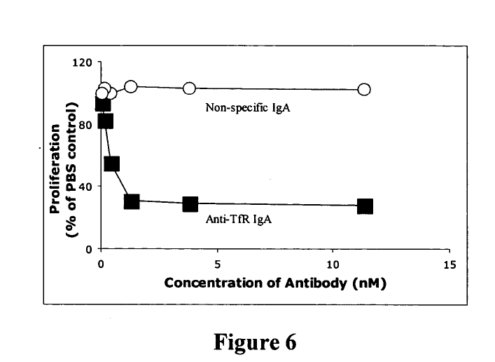

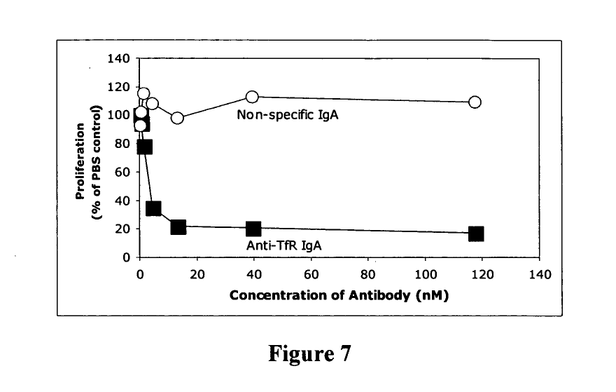

Malignant tumor cells overexpress transferrin receptors (TfRs). To determine the effect of anti-human transferrin receptor, two hematopoietic cancer cell lines were incubated with anti-TfR IgA antibody. First, human ARH-77 cells were treated with indicated concentrations of anti-TfR IgA and non-specific IgA for 48 hours (FIG. 6). The cells were then cultured in the presence of [³H]thymidine for an additional 24 hours. The cells were harvested and [³]thymidine incorporation determined. Each value is a mean of four replicate values expressed as the % of control (cells treated with buffer alone) mean. Results from two independent experiments were averaged in this experiment. FIG. 6 indicates that the Anti-TfR IgA antibody exhibits antproliferative activity on human ARH-77 cells. Next, human myeloma IM-9 cells were treated with indicated concentrations of anti-TfR IgA and non-specific IgA for 96 hours (FIG. 7). Proliferation was determined using CellTiter 96 AQueous Non-Radioactive Cell Proliferation Assay (Promega). Each value is a mean of four replicate values expressed as the % of control (cells treated with buffer alone) mean. At a concentration of 4.5 nM, anti-TfR IgA inhibited 65% of proliferation of ARH-77 and 18 nM of anti-TfR inhibited 80% of IM-9 proliferation. In contrast, no inhibition was observed in presence of a non-specific IgA (FIGS. 6 and 7).

### EXAMPLE 7

### Anti-Human Transferrin Receptor IgA Induces Apoptosis in the Hematopoietic Cancer Cell Lines ARH-77 and IM-9

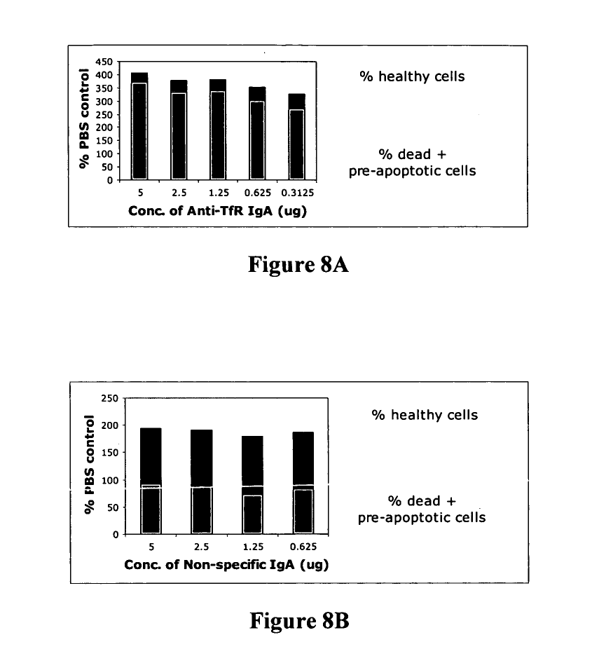

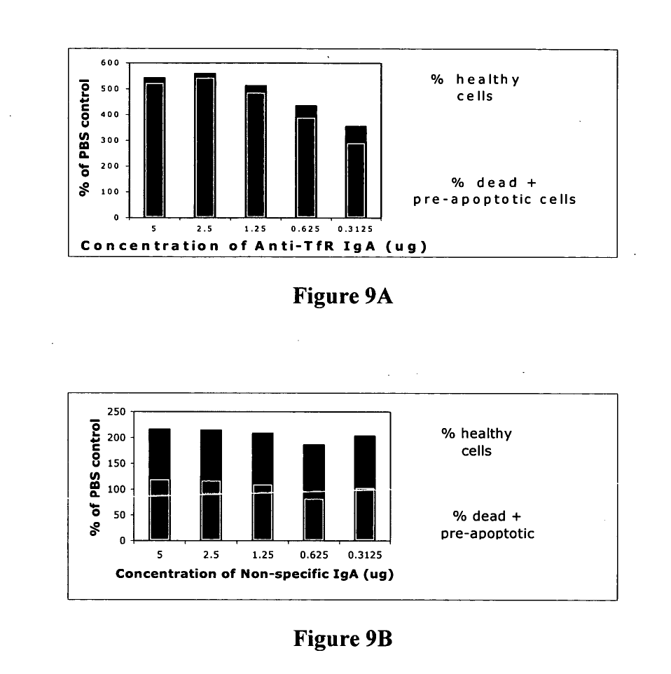

To determine if cell death by apoptosis followed inhibition of proliferation, cells treated with varying concentration of anti-TfR IgA were assayed for annexin V affinity staining. Human ARH-77 cells were incubated with buffer alone, anti-TfR IgA (FIG. 8A), and non-specific IgA (FIG. 8B) for 96 hours. The cells were then washed, stained with AlexaFluor 488 Annexin V and Propidium Iodide, and analyzed by flow cytometry. Each value expressed as the % of control (cells treated with buffer alone). In addition, human myeloma IM-9 cells were incubated with buffer alone, anti-TfR IgA (FIG. 9A), and non-specific IgA (FIG. 9B) for 96 hours. The cells were then washed, stained with AlexaFluor 488 Annexin V and Propidium Iodide, and analyzed by flow cytometry. Each value expressed as the % of control (cells treated with buffer alone). Following treatment with anti-TfR IgA, there was a dramatic increase in the staining with annexin V, suggesting that apoptosis had been initiated in all cells (FIGS. 8A and 8b; FIG. 9A and 9B).

### EXAMPLE 8

### Purification of IgA from Culture Supernatants

IgA was purified from culture supernatants by affinity chromatography on a column containing goat anti-human IgA (Sigma Immuno. Chem., St. Louis, Mo.) and immobilized on Sepharose 4B. Unbound proteins were washed with PBS. Bound IgA was then eluted using 0.1M glycine pH 2.5. To minimize the effect of low pH on IgA, the pH of the eluted protein was immediately adjusted to pH 7.2 using IM Tris, pH 8.0. The eluted proteins were then concentrated and dialyzed against PBS. Protein concentrations were determined with a combination of the bicinchoninic acid assay (Pierce, Rockford, Ill.) and comparison with a standard of known concentration followed by SDS-PAGE and staining with Coomassie blue. Monomeric IgA was separated from dimeric IgA by gel filtration on two Superose 6 columns (Amersham Pharmacia Biotech, Piscataway, N.J.) in series in PBS. Chintalacharuvu et al., Mol. Immunol. 30:19 (1993). The covalent structure of the purified proteins was confirmed by SDS-PAGE. Fractions were pooled and the structure of the concentrated material confirmed by SDS-PAGE.

For large cultures, transfectants may be grown in roller bottles in IMDM supplemented with 1% alpha calf serum (Hyclone, Logan, Utah) and glutaiax. Supernatants may be centrifuged at 5,000 rpm, filtered to remove any cells and cell debris and supplemented with 10 mM phosphate buffer, pH 6.8, 0.45 NaCl, 0.02M EDTA and 0.02% NaN₃ and stored at 4° C. until protein purification.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention.

What is claimed is:

1. A compound comprising:
1) a heavy chain constant region and a light chain constant region of a human antibody; and
2) a heavy chain variable region and a light chain variable region that recognizes the human transferrin receptor.

2. The compound of claim 1, wherein the heavy chain constant region and the light chain constant region comprise human IgA.

3. The compound of claim 1, wherein the heavy chain constant region and the light chain constant region comprise human IgM.

4. The compound of claim 1, wherein the heavy chain constant region and the light chain constant region comprise human polymeric IgG.

5. The compound of claim 1, wherein the heavy chain variable region and light chain variable region are murine.

6. A pharmaceutical composition comprising the compound of claim 1 in combination with a pharmaceutically acceptable carrier.

7. A method of treating a malignancy that expresses the human transferrin receptor in an individual, comprising administering the pharmaceutical composition of claim 6 to the individual, in a therapeutically effective amount.

8. The method of claim 7, wherein the malignancy is selected from the group consisting of multiple myeloma, leukemia and lymphoma.

9. A method of causing apoptosis or cell death in cells expressing the human transferrin receptor, comprising contacting said cell with the compound of claim 1.

10. The method of claim 9, wherein the heavy chain constant region and the light chain constant region of said compound comprise human IgA.

11. The method of claim 9, wherein the heavy chain constant region and the light chain constant region of said compound comprise human IgM.

12. The method of claim 9, wherein the heavy chain constant region and the light chain constant region of said compound comprise human polymeric IgG.

13. The method of claim 9, wherein the heavy chain variable region and light chain variable region of said compound are murine.

14. The method of claim 9, wherein said cells comprise malignant cells.

15. The method of claim 14, wherein said malignant cells are selected from the group consisting of multiple myeloma cells, leukemia cells and lymphoma cells.

16. A therapeutic composition for causing apoptosis or cell death in cells expressing the transferrin receptor on their surface, said composition comprising the compound of claim 1.

17. The therapeutic composition of claim 16, wherein the heavy chain constant region and the light chain constant region of said compound comprise human IgA.

18. The therapeutic composition of claim 16, wherein the heavy chain constant region and the light chain constant region of said compound comprise human IgM.

19. The therapeutic composition of claim 16, wherein the heavy chain constant region and the light chain constant region of said compound comprise human polymeric IgG.

20. The therapeutic composition of claim 16, wherein the heavy chain variable region and light chain variable region of said compound are murine.

21. The therapeutic composition of claim 16, wherein said cells comprise malignant cells.

22. The therapeutic composition of claim 21, wherein said malignant cells are selected from the group consisting of multiple myeloma cells, leukemia cells and lymphoma cells.

23. A method of inhibiting proliferation of cells expressing the human transferrin receptor, comprising contacting said cell with the compound of claim 1.

24. The method of claim 23, wherein the heavy chain constant region and the light chain constant region of said compound comprise human IgA.

25. The method of claim 23, wherein the heavy chain constant region and the light chain constant region of said compound comprise human IgM.

26. The method of claim 23, wherein the heavy chain constant region and the light chain constant region of said compound comprise human polymeric IgG.

27. The method of claim 23, wherein the heavy chain variable region and light chain variable region of said compound are murine.

28. The method of claim 23, wherein said cells comprise malignant cells.

29. The method of claim 28, wherein said malignant cells are selected from the group consisting of multiple myeloma cells, leukemia cells and lymphoma cells.

30. A therapeutic composition for inhibiting proliferation of cells expressing the transferrin receptor on their surface, said composition comprising the compound of claim 1.

31. The therapeutic composition of claim 30, wherein the heavy chain constant region and the light chain constant region of said compound comprise human IgA.

32. The therapeutic composition of claim 30, wherein the heavy chain constant region and the light chain constant region of said compound comprise human IgM.

33. The therapeutic composition of claim 30, wherein the heavy chain constant region and the light chain constant region of said compound comprise human polymeric IgG.

34. The therapeutic composition of claim 30, wherein the heavy chain variable region and light chain variable region of said compound are murine.

35. The therapeutic composition of claim 30, wherein said cells comprise malignant cells.

36. The therapeutic composition of claim 35, wherein said malignancy cells are selected from the group consisting of multiple myeloma cells, leukemia cells and lymphoma cells.

37. A method for autologous hematopoietic cell transplantation in a subject suffering from multiple myeloma, the method comprising:
(1) removing the hematopoietic progenitor cell population from the subject;
(2) treating the cell population with the compound of claim 1; and
(3) transplanting the treated cell population from step (2) into the subject.

38. The method of claim 37, wherein the heavy chain constant region and the light chain constant region of said compound comprise human IgA.

39. The method of claim 37, wherein the heavy chain constant region and the light chain constant region of said compound comprise human IgM.

40. The method of claim 37, wherein the heavy chain constant region and the light chain constant region of said compound comprise human polymeric IgG.

41. The method of claim 37, wherein the heavy chain variable region and light chain variable region of said compound are murine.
